(19) United States
(12) Patent Application Publication
(10) Pub. No.: US 2009/0259290 A1
(43) Pub. Date: Oct. 15, 2009

(54) Fenestration Segment Stent-Graft and Fenestration Method

(75) Inventors: Walter Bruszewski, Guerneville, CA (US); Trevor Greenan, Santa Rosa, CA (US); Jack Chu, Santa Rosa, CA (US); David Erickson, Memphis, TN (US); Jonathan Morris, Santa Rosa, CA (US); Prema Ganesan, Oakland, CA (US); Curtis Hanson, San Marcos, CA (US); Matthew Rust, North Vancouver (CA); Charles Thomas, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/102,320

(22) Filed: Apr. 14, 2008

(51) Int. Cl. A61F 2/06

(52) U.S. Cl. 623/1.13

(57) ABSTRACT

A method includes deploying a fenestration segment stent-graft into a main vessel such that a fenestration section of the fenestration segment stent-graft covers a first branch vessel emanating from the main vessel. The fenestration segment stent-graft includes a proximal section, a distal section, and the fenestration section attached to and between the proximal section and the distal section. The fenestration section has a greater resistance to tearing than the proximal section and the distal section facilitating formation of a collateral opening aligned with the branch vessel in the fenestration section.

### BACKGROUND OF THE INVENTION

1. Field of the Invention

The present invention relates to an intra-vascular device and method. More particularly, the present invention relates to a device for treatment of intra-vascular diseases.

2. Description of Related Art

A conventional main (vessel) stent-graft typically includes a radially expandable reinforcement structure, formed from a plurality of annular stent rings, and a cylindrically shaped layer of graft material, sometimes called graft cloth, defining a lumen to which the stent rings are coupled. Main stent-grafts are well known for use in tubular shaped human vessels.

To illustrate, endovascular aneurysmal exclusion is a method of using a main stent-graft to exclude pressurized fluid flow from the interior of an aneurysm, thereby reducing the risk of rupture of the aneurysm and the associated invasive surgical intervention.

Main stent-grafts with custom side openings are sometimes fabricated to accommodate the particular vessel structure of each individual patient. Specifically, as the location of branch vessels emanating from a main vessel, e.g., the vessel having the aneurysm, varies from patient to patient, main stent-grafts are fabricated with side openings customized to match the position of the branch vessels of the particular patient. However, custom fabrication of main stent-grafts is relatively expensive and time consuming.

To avoid custom fabrication of main stent-grafts, side openings in the main stent-graft may be formed in situ. Illustratively, the main stent-graft is placed in the main vessel, e.g., the aorta, to exclude an aneurysm. Side openings are made in situ to correspond to and perfuse the branch vessels.

However, deployment of the main stent-graft temporarily interrupts perfusion to the branch vessels until the side openings are formed in the main stent-graft. In various applications, perfusion to the branch vessels cannot be interrupted for any significant interval of time. Accordingly, the formation of side openings in a main stent-graft in situ is a complicated and risky procedure.

### SUMMARY OF THE INVENTION

A method includes deploying a fenestration segment stent-graft into a main vessel such that a fenestration section of the fenestration segment stent-graft covers a first branch vessel emanating from the main vessel. The fenestration segment stent-graft includes a proximal section, a distal section, and the fenestration section attached to and between the proximal section and the distal section. The fenestration section has a greater resistance to tearing than the proximal section and the distal section facilitating formation of a collateral opening aligned with the branch vessel in the fenestration section.

In one example, the fenestration section is permeable thus allowing the branch vessel to be perfused through the fenestration section. In this manner, the branch vessel is perfused through the fenestration section during the entire procedure of deploying and fenestrating the fenestration segment stent-graft. Accordingly, the complexity and risk of the procedure is reduced.

Embodiments are best understood by reference to the following detailed description when read in conjunction with the accompanying drawings.

### BRIEF DESCRIPTION OF THE DRAWINGS

FIG. 1 is a perspective view of a fenestration segment stent-graft in accordance with one embodiment;

FIG. 2 is a cross-sectional view of the fenestration segment stent-graft of FIG. 1;

FIGS. 3, 4, 5, 6, and 7 are enlarged views of a portion of a fenestration section of the fenestration segment stent-graft of FIGS. 1 and 2 in accordance with various examples;

FIG. 8 is a cross-sectional view of a vessel assembly including the fenestration segment stent-graft of FIGS. 1 and 2 in accordance with one example;

FIG. 9 is an enlarged cross-sectional view of the vessel assembly of FIG. 8 during fenestration of the fenestration segment stent-graft;

FIGS. 10, 11 are enlarged cross-sectional views of the vessel assembly of FIG. 9 during further stages of fenestration of the fenestration segment stent-graft;

FIG. 12 is a simplified perspective view of an expandable cutting strut device in the radially expanded configuration of FIG. 11;

FIG. 13 is a cross-sectional view of a strut of the expandable cutting strut device along the line XIII-XIII of FIG. 12 in accordance with one example;

FIGS. 14, 15 are cross-sectional views of struts similar to the strut of FIG. 13 in accordance with other examples;

FIGS. 16, 17 are enlarged cross-sectional views of the vessel assembly of FIG. 11 during further stages of fenestration of the fenestration segment stent-graft;

FIG. 18 is a schematic view of a vessel assembly including a fenestration segment stent-graft similar to the fenestration segment stent-graft of FIGS. 1 and 2 prior to fenestration in accordance with another example; and

FIGS. 19, 20, 21 are schematic views of vessel assemblies including fenestration segment stent-grafts similar to the fenestration segment stent-graft of FIGS. 1 and 2 prior to fenestration in accordance with other examples.

Common reference numerals are used throughout the drawings and detailed description to indicate like elements.

### DETAILED DESCRIPTION

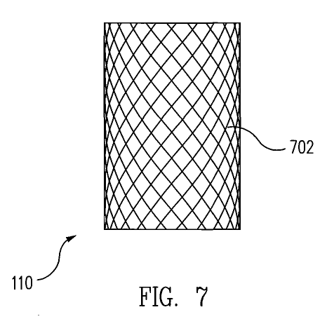

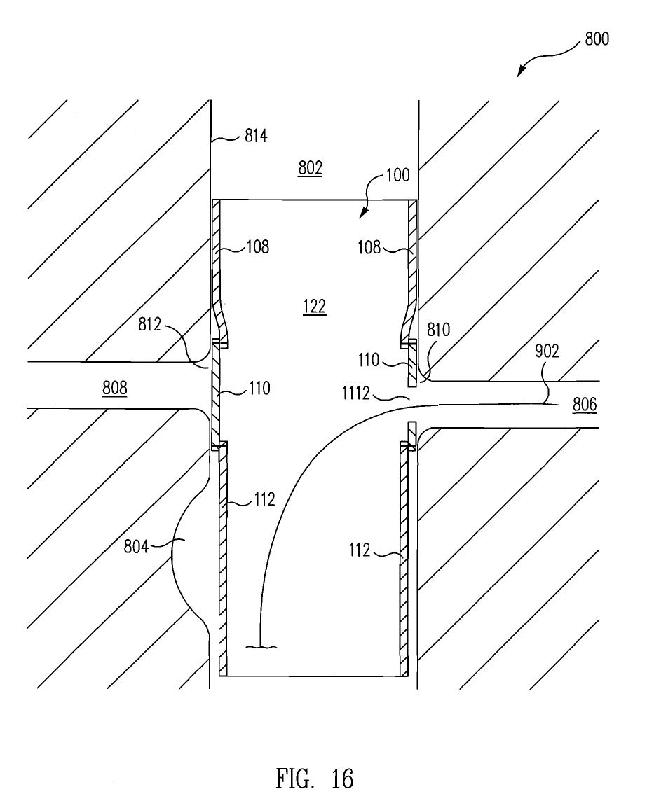

Referring to FIG. 8, a method includes deploying a fenestration segment stent-graft 100 into a main vessel 802 such that a fenestration section 110 of fenestration segment stent-graft 100 covers a first branch vessel 806 emanating from main vessel 802. Fenestration segment stent-graft 100 includes a proximal section 108, a distal section 112, and fenestration section 110 attached to and between proximal section 108 and distal section 112. Fenestration section 110 has a greater resistance to tearing than proximal section 108 and distal section 112 facilitating formation of a collateral opening 1112 (FIG. 16) aligned with branch vessel 806 in fenestration section 110.

In one example, fenestration section 110 is permeable thus allowing branch vessel 806 to be perfused through fenestration section 110. In this manner, branch vessel 806 is perfused through fenestration section 110 during the entire procedure of deploying and fenestrating fenestration segment stent-graft 100. Accordingly, the complexity and risk of the procedure is reduced.

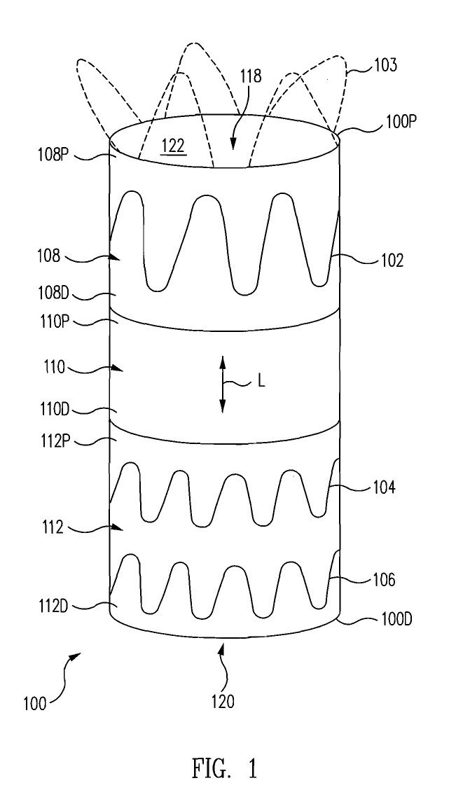

More particularly, FIG. 1 is a perspective view of a fenestration segment stent-graft 100, e.g., an abdominal aortic stent-graft, in accordance with one embodiment. Referring now to FIG. 1, fenestration segment stent-graft 100 includes stent rings 102, 104, 106. Illustratively, stent rings 102, 104, 106 are self-expanding stent rings, e.g., formed of Nitinol.

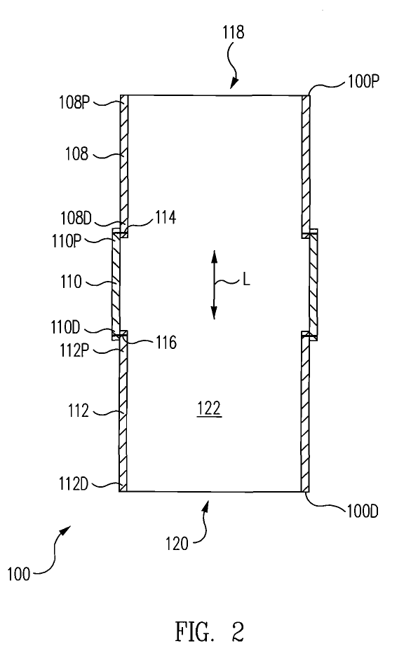

FIG. 2 is a cross-sectional view of fenestration segment stent-graft 100 of FIG. 1. In FIG. 2, stent rings 102, 104, 106 are not illustrated for clarity of presentation.

Referring now to FIGS. 1 and 2 together, fenestration segment stent-graft 100 includes a proximal section 108, a fenestration section 110, and a distal section 112, sometimes called a proximal segment, a fenestration segment and a distal segment, respectively. Fenestration section 110 is attached to and between proximal section 108 and distal section 112.

As used herein, the proximal end of a prosthesis such as a stent-graft is the end closest to the heart via the path of blood flow whereas the distal end is the end furthest away from the heart during deployment. In contrast and of note, the distal end of the catheter is usually identified to the end that is farthest from the operator (handle) while the proximal end of the catheter is the end nearest the operator (handle).

For purposes of clarity of discussion, as used herein, the distal end of the catheter is the end that is farthest from the operator (the end furthest from the handle) while the distal end of the prosthesis is the end nearest the operator (the end nearest the handle), i.e., the distal end of the catheter and the proximal end of the stent-graft are the ends furthest from the handle while the proximal end of the catheter and the distal end of the stent-graft are the ends nearest the handle. However, those of skill in the art will understand that depending upon the access location, the stent-graft and delivery system description may be consistent or opposite in actual usage.

Proximal section 108 includes a proximal end 108P and a distal end 108D. Fenestration section 110 includes a proximal end 110P and a distal end 110D. Distal end 108D of proximal section 108 is attached to proximal end 110P of fenestration section 110 by an attachment means 114. Illustratively, attachment means 114 is stitching, adhesive, thermal bonding, or other attachment between proximal section 108 and fenestration section 110.

Distal section 112 includes a proximal end 112P and a distal end 112D. Proximal end 11 2P of distal section 112 is attached to distal end 110D of fenestration section 110 by an attachment means 116. Illustratively, attachment means 116 is stitching, adhesive, thermal bonding, or other attachment between fenestration section 110 and distal section 112.

Fenestration segment stent-graft 100 includes a proximal main opening 118 at a proximal end 10OP of fenestration segment stent-graft 100 and a distal main opening 120 at a distal end 100D of fenestration segment stent-graft 100. Further, fenestration segment stent-graft 100 includes a longitudinal axis L. A main lumen 122 is defined by fenestration segment stent-graft 100 and extends generally parallel to longitudinal axis L and between proximal main opening 118 and distal main opening 120 of fenestration segment stent-graft 100.

Proximal section 108, fenestration section 110, and distal section 112 are cylindrical having a substantially uniform diameter.

In one embodiment, proximal section 108 is a first cylindrical piece of graft material, e.g., woven graft cloth. Distal section 112 is a second cylindrical piece of graft material, e.g., woven graft cloth.

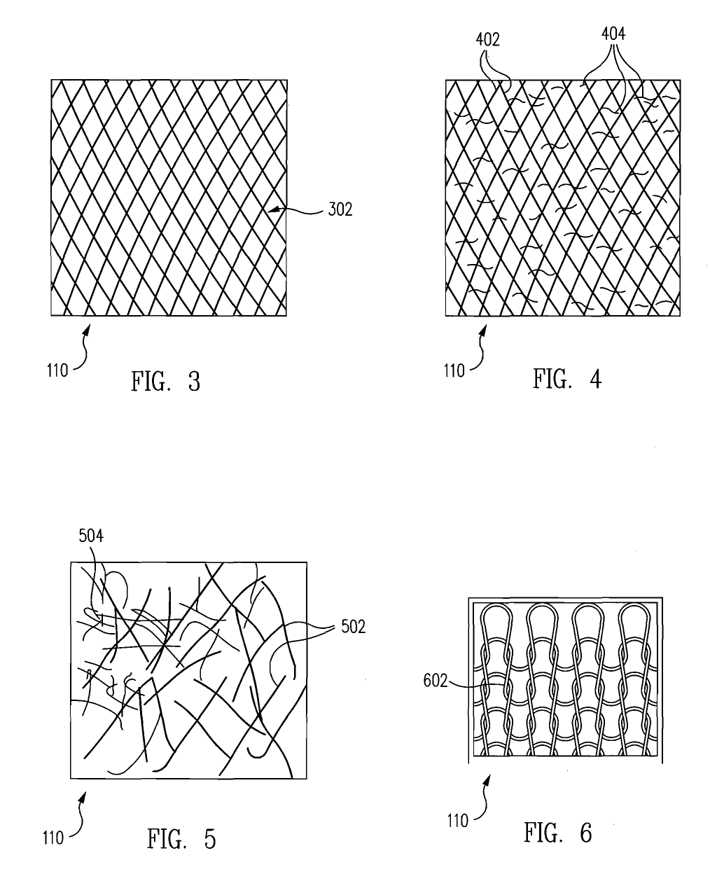

Fenestration section 110 is formed from a third cylindrical piece of material such as those discussed below with reference to FIGS. 3, 4, 5, 6 and 7.

In one particular example as discussed below with reference to FIG. 6, fenestration section 110 is formed of knit cloth. Because of the extra mobility of the yarn in the knit cloth, puncture and dilation of fenestration section 110 as discussed below results in little or no tear propagation and creates a compliant sealing region around the puncture.

This is in contrast to a woven graft cloth, in which a tear in at least one direction is produced when the woven graft cloth is punctured and dilated. Further, in a woven graft cloth, the tear is typically propagated when a device such as a covered stent is inserted and loaded into the opening formed in the woven graft cloth. Specifically, fenestration section 110 formed of knit cloth has a greater resistance to tearing than proximal section 108/distal section 112, which are formed of woven graft cloth.

In other examples as discussed below with reference to FIGS. 3, 4, and 5, fenestration section 110 is formed of a porous material. As set forth below, when fenestration section 110 is deployed over branch vessels, the branch vessels are nevertheless perfused through porous fenestration section 110 during the entire procedure of deploying fenestration segment stent-graft 100. Stated another way, perfusion to the branch vessels is not interrupted for any significant interval of time. Accordingly, the complexity and risk of the procedure is reduced. Further, clotting of fenestration section 110, i.e., after formation of collateral openings in fenestration section 110, decrease the permeability of fenestration section 110, i.e., increases the sealing of fenestration section 110, over time.

Referring still to FIGS. 1 and 2 together, stent ring 102 is attached, e.g., sewn, to proximal section 108. Similarly, stent rings 104, 106 are attached, e.g., sewn, to distal section 112.

Stent rings 102, 104, 106, are self-expanding facilitating expansion, fixation, and sealing of fenestration segment stent-graft 100 into the main vessel as discussed further below. In another example, a fenestration segment stent-graft similar to fenestration segment stent-graft 100 is formed with stent rings that are balloon expanded facilitating fixation and sealing of the fenestration segment stent-graft into the main vessel. Fenestration section 110 has an absence of stent rings.

Although three stent rings 102, 104, 106 are illustrated, in other examples, a fenestration segment stent-graft similar to fenestration segment stent-graft 100 is formed with more or less than three stent rings or other self-expanding members. For example, a stent ring 103 as illustrated by the dashed lines in FIG. 1 is attached to proximal section 108 and extends proximally therefrom. Stent ring 103 is not illustrated in the remaining figures.

Fenestration section 110 is formed of a porous material which facilitates fenestration (formation of openings) in situ while at the same time avoids formation of a rent, sometimes called a tear. Further, the porous material of fenestration section 110 facilitates clotting and sealing over time. Various examples of materials of fenestration section 110 are set forth below with reference to FIGS. 3, 4, 5, 6, and 7.

FIG. 3 is an enlarged view of a portion of fenestration section 110 in accordance with one example. As illustrated, fenestration section 110 is formed of a graft material formed of loose woven fibers 302. In accordance with this example, loose woven fibers 302 are continuous fibers. In one example, fenestration section 110 is formed of loosely woven PET graft material. In accordance with this example, the graft material is formed by weaving, and includes warp though which the weft is woven. The weave is loose, allowing the fibers to be readily moved facilitating fenestration and dilation of fenestration section 110.

FIG. 4 is an enlarged view of a portion of fenestration section 110 in accordance with another example. As illustrated, fenestration section 110 is formed of a graft material formed of loose woven fibers 402 and includes a velour 404. Velour 404, e.g., loose loops of fiber, forms a napped surface. Velour 404 promotes formation of thrombus on fenestration section 110.

FIG. 5 is an enlarged view of a portion of fenestration section 110 in accordance with another example. As illustrated, fenestration section 110 is formed of a graft material formed of loose random fibers 502 and includes a velour 504. Loose random fibers 502 are randomly oriented with respect to one another. The fibers are loose and randomly oriented, allowing the fibers to be readily moved facilitating fenestration and dilation of fenestration section 110.

Velour 504, e.g., loose loops of fiber, forms a napped surface. Velour 504 promotes formation of thrombus on fenestration section 110. In another embodiment, a fenestration section similar to fenestration section 110 is formed of a graft material formed of loose random fibers similar to loose random fibers 502 but having an absence of velour. In another example, fenestration section 110 is formed of randomly oriented PET graft material.

FIG. 6 is an enlarged view of a portion of fenestration section 110 in accordance with another example. As illustrated, fenestration section 110 is formed of a graft material formed of knit cloth 602. In accordance with this example, the graft material is formed of loops called stitches that are pulled through each other. These stitches are readily moved and stretched facilitating fenestration and dilation of fenestration section 110.

Knit cloth 602 imparts stretchability and mobility for variations in branch vessel, e.g., renal artery, geometry. Further, knit cloth 602 can be punctured without tearing. Further, knit cloth 602 creates a seal around a branch prosthesis, sometimes called a renal artery branch connection, such as branch prosthesis 1714 illustrated and discussed below in reference to FIG. 17.

In one example, knit cloth 602 is impregnated with an elastomer such as silicone, polyurethane, or other elastomer. By impregnating knit cloth 602 with an elastomer, tear propagation in knit cloth 602 is prevented while sealing around the branch prosthesis is enhanced.

FIG. 7 is an enlarged view of a portion of fenestration section 110 in accordance with another example. As illustrated, fenestration section 110 is formed of graft material formed of a tubular braid 702. In accordance with this example, the graft material is a structure formed by intertwining strands, with each strand functionally equivalent in zigzagging forward through the overlapping mass of the other strands.

In another example, fenestration section 110 is formed of a low density monofilament graft material that allow perfusion acutely but will clot and seal over time as anticoagulation therapy is reversed. Further, an anti-thrombogenic coating can be applied over the monofilament fibers to allow better initial perfusion through a dense weave while facilitating more effective subsequent sealing. For example, the anti-thrombogenic coating is a heparin coating that degrades quickly, e.g., in 2-24 hours. In another example, the anti-thrombogenic coating is applied over a thrombogenic coating applied over the monofilament fibers. The anti-thrombogenic coating degrades to reveal the thrombogenic coating further enhancing sealing of the low density monofilament graft material.

In another example, only a thrombogenic coating is applied such as thrombin, fibrin, or other thrombogenic material to promote thrombus and reduce permeability of fenestration section 110.

In other examples, fenestration section 110 if formed of Polyethylene terephthalate (PET), e.g., woven PET, expanded Polytetrafluoroethylene (ePTFE), e.g., extruded or casted ePTFE or high porosity ePTFE graft material.

FIG. 8 is a cross-sectional view of a vessel assembly 800 including fenestration segment stent-graft 100 of FIGS. 1 and 2 in accordance with one example. Referring now to FIG. 8, a main vessel 802, e.g., the aorta, includes an aneurysm 804. Fenestration segment stent-graft 100, sometimes called a prosthesis, is deployed into main vessel 802 to exclude aneurysm 804 using any one of a number of techniques well known to those of skill in the art.

Emanating from main vessel 802 is a first branch vessel 806 and a second branch vessel 808, sometimes called visceral branches of the abdominal aorta. The location of branch vessels 806, 808 vary from patient to patient. Examples of branch vessels include the renal arteries (RA) and the superior mesenteric artery (SMA).

Fenestration segment stent-graft 100 is deployed such that fenestration section 110 is aligned with branch vessels 806, 808. Stated another way, fenestration segment stent-graft 100 is deployed such that fenestration section 110 covers ostia (plural of ostium) 810, 812 of branch vessels 806, 808, respectively.

Proximal section 108 is located proximally to ostia 810, 812 of branch vessels 806, 808. Accordingly, fenestration segment stent-graft 100 is deployed with fixation and sealing superior to branch vessels 806, 808. Distal section 112 is located distally to ostia 810, 812 of branch vessels 806, 808, respectively.

Stent rings 102, 104, 106 (see FIG. 1) are radially expandable reinforcement structures that self-expand into a vessel wall 814 of main vessel 802 thus anchoring fenestration segment stent-graft 100 in place. Once anchored within main vessel 802, blood flows through main lumen 122 and more generally through fenestration segment stent-graft 100 thus excluding aneurysm 804.

Further, permeable fenestration section 110 allows branch vessels 806, 808 to be perfused through fenestration section 110. More particularly, the pressure inside of fenestration segment stent-graft 100 is greater than the pressure within branch vessels 806, 808. Due to this pressure differential, blood flows through fenestration section 110, which is permeable.

In this manner, branch vessels 806, 808 are perfused through fenestration section 110 during the entire procedure of deploying and fenestrating fenestration segment stent-graft 100. Stated another way, perfusion to branch vessels 806, 808 is not interrupted for any significant interval of time. Accordingly, the complexity and risk of the procedure is reduced.

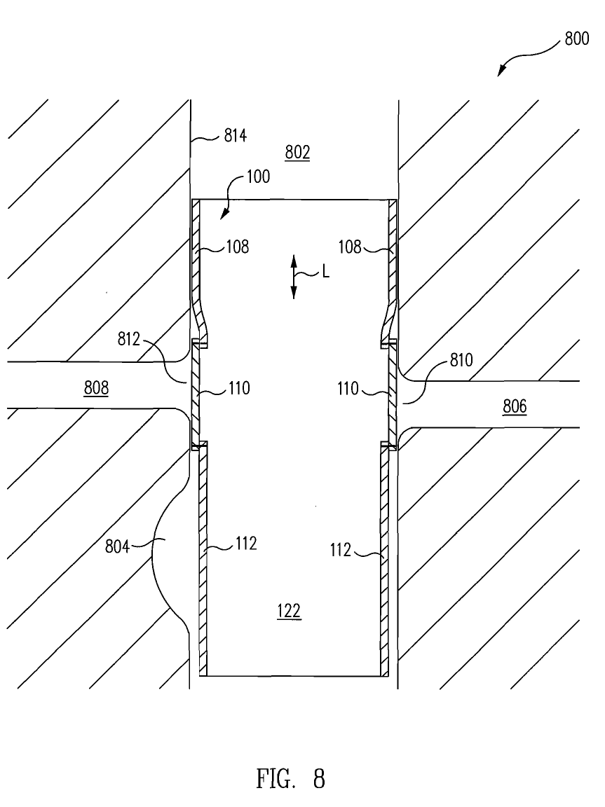

FIG. 9 is an enlarged cross-sectional view of vessel assembly 800 of FIG. 8 during fenestration of fenestration segment stent-graft 100. Referring now to FIG. 9, to form a side opening, sometimes called a collateral opening, in fenestration segment stent-graft 100, and more particularly, fenestration section 110, corresponding to (at) branch vessel 806, a steerable guide wire 902 is advanced to the location of branch vessel 806. Steerable guide wires similar to steerable guide wire 902 are well known to those of skill in the art.

Once located at branch vessel 806, outward force on guide wire 902 causes guide wire 902 to fenestrate (penetrate) fenestration section 110 thus forming a guide wire hole 904 in fenestration section 110 in alignment with branch vessel 806. Accordingly, guide wire 902 extends from inside main lumen 122 of fenestration segment stent-graft 100, though guide wire hole 904 in fenestration section 110, and into branch vessel 806.

In one example, fenestration section 110 is initially pierced with a sharp hollow needle, and guide wire 902 is advanced through the needle and into branch vessel 806. The needle is removed resulting in the assembly as illustrated in FIG. 9.

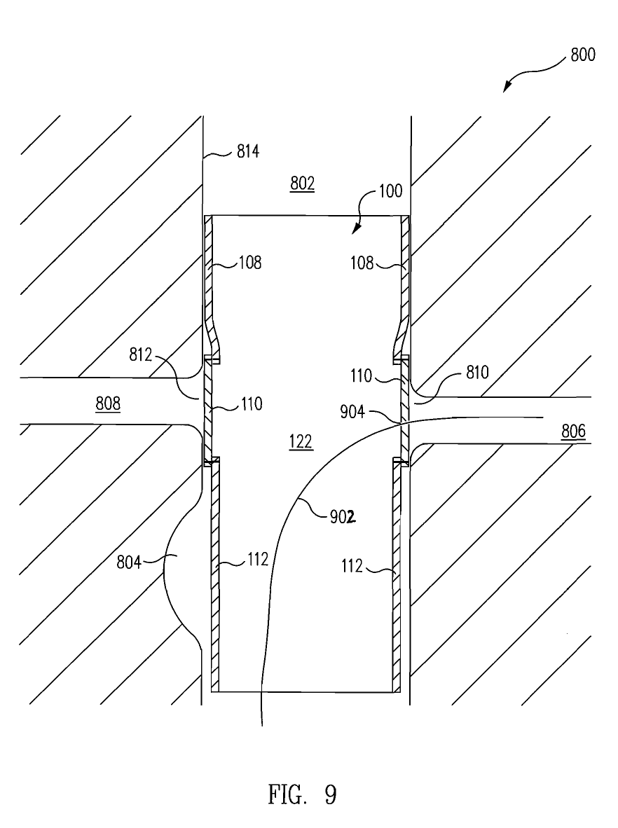

FIG. 10 is an enlarged cross-sectional view of vessel assembly 800 of FIG. 9 during a further stage of fenestration of fenestration segment stent-graft 100. Referring now to FIGS. 9 and 10 together, a cutting strut catheter 1000 is advanced over guide wire 902 and located inside of fenestration section 110.

Cutting strut catheter 1000 includes a tapered tip 1002, an inner member 1004, an expandable cutting strut device 1006, and an outer sheath 1008. Tapered tip 1002 is mounted on the distal end 1004D of inner member 1004. Tapered tip 1002 and inner member 1004 define a guidewire lumen therein though which guidewire 902 extends.

Tapered tip 1002 and distal end 1008D of outer sheath 1008 include tapered outer surfaces facilitating advancement of cutting strut catheter 1000 through guidewire hole 904 in fenestration section 110. Cutting strut catheter 1000 is advanced through guidewire hole 904 thus dilating (enlarging, sometimes called increasing in diameter) guidewire hole 904 (FIG. 9) to form a dilated guidewire hole 1010 (FIG. 10). Dilated guidewire hole 1010 has a larger diameter than guidewire hole 904. As set forth above, fenestration section 110 is formed of a material that facilitates dilation of guidewire hole 904 without formation of a tear, sometimes called rent, in fenestration section 110.

In one embodiment, expandable cutting strut device 1006 is a self-expanding device, e.g., formed of Nitinol (NiTi alloy). In accordance with this example, expandable cutting strut device 1006 is radially constrained within the lumen defined by outer sheath 1008.

FIG. 11 is an enlarged cross-sectional view of vessel assembly 800 of FIG. 10 during a further stage of fenestration of fenestration segment stent-graft 100. Referring to FIG. 11, outer sheath 1008 is retracted thus exposing expandable cutting strut device 1006. In one example, upon being exposed, expandable cutting strut device 1006 radially self-expands into fenestration section 110 thus enlarging (cutting and/or dilating) dilated guidewire hole 1010 (FIG. 10) into a collateral opening 1112. As set forth above, fenestration section 110 is formed of a material that facilitates enlargement of dilated guidewire hole 1010 without formation of a tear in fenestration section 110.

In another example, upon being exposed, expandable cutting strut device 1006, e.g., stainless steel, is radially expanded by a dilation balloon inside of expandable cutting strut device 1006. Radial expansion of expandable cutting strut device 1006 into fenestration section 110 enlarges dilated guidewire hole 1010 (FIG. 10) into a collateral opening 1112.

Although use of cutting strut catheter 1000 to form collateral opening 1112 is set forth herein, in other examples, a small hole, e.g., guidewire hole 904, is dilated to form collateral opening 1112 by passing an enlarging structure, e.g., a dilator, through the small hole.

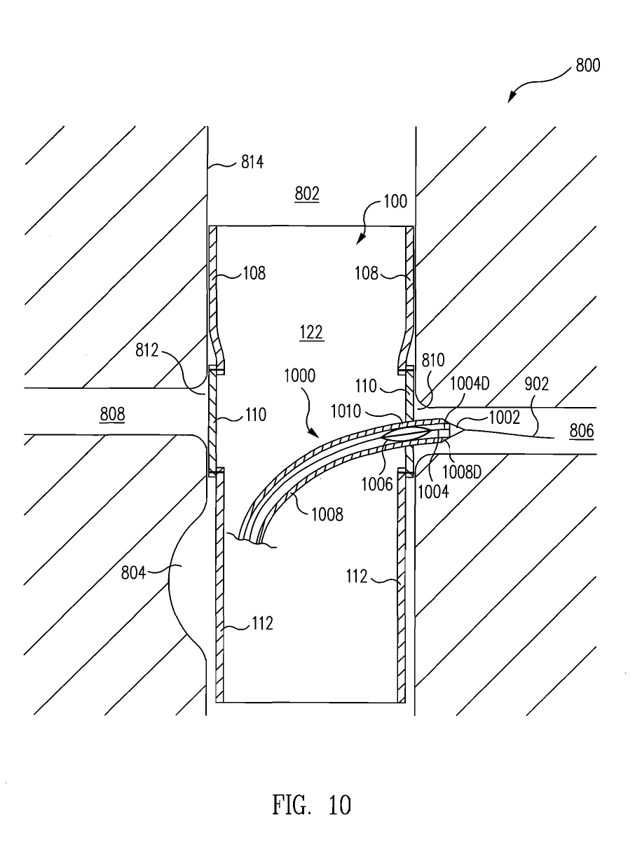

FIG. 12 is a simplified perspective view of expandable cutting strut device 1006 in the radially expanded configuration of FIG. 11. Referring now to FIGS. 11 and 12 together, expandable cutting strut device 1006 includes a plurality of struts 1214, e.g., strips of metal. Struts 1214 are connected together at a distal end 1006D and at a proximal end 1006P of expandable cutting strut device 1006. Accordingly, expandable cutting strut device 1006 increases in diameter between distal end 1006D and proximal end 1006P having a greatest outer diameter therebetween.

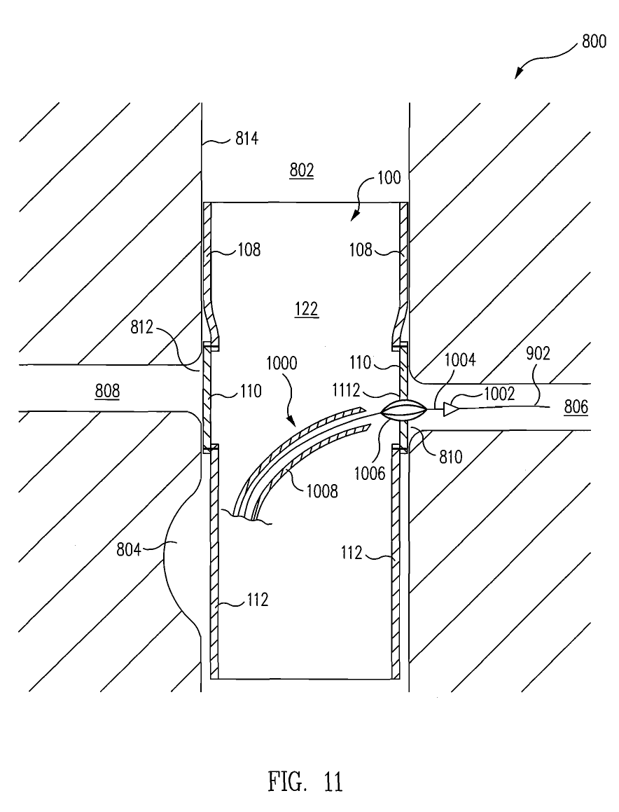

In one example, struts 1214 are formed with sharp edges to facilitate cutting of fenestration section 110 and formation of collateral opening 1112. FIG. 13 is a cross-sectional view of a strut 1214 of expandable cutting strut device 1006 along the line XIII-XIII of FIG. 12 in accordance with one example. Referring now to FIGS. 12 and 13 together, strut 1214 includes a radially outward projecting sharp taper 1302. Taper 1302 decreasingly tapers in width radially outward to a sharp edge 1304.

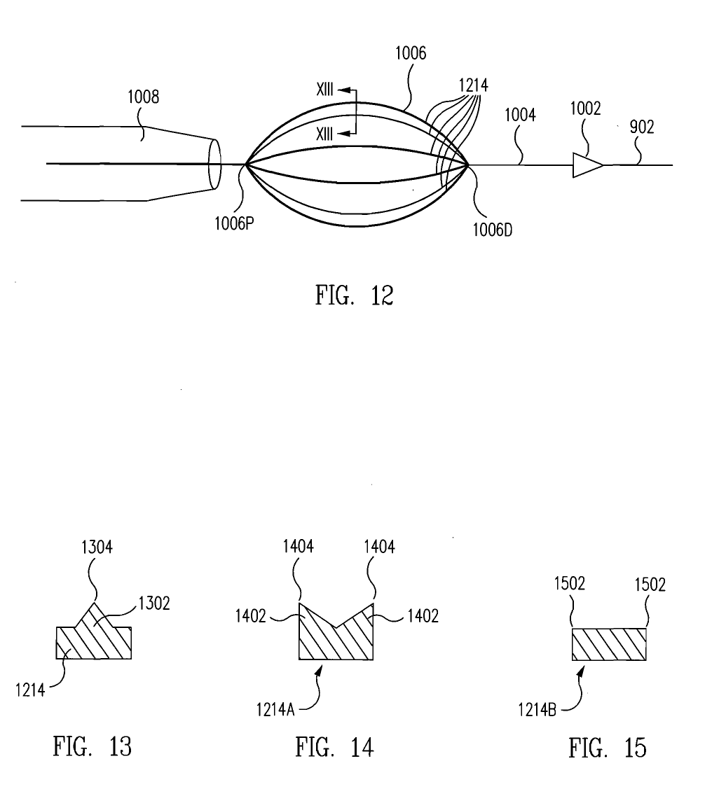

FIG. 14 is a cross-sectional view of a strut 1214A similar to strut 1214 of FIG. 13 in accordance with another example. Referring now to FIG. 14, strut 1214A includes a pair of radially outward projecting sharp tapers 1402. Tapers 1402 decreasingly tapers in width radially outward to sharp edges 1404.

FIG. 15 is a cross-sectional view of a strut 1214B similar to strut 1214 of FIG. 13 in accordance with another example. Referring now to FIG. 15, strut 1214B is a rectangular shaped member having a pair of sharpened edges 1502. Edges 1502 are the radially outward edges of strut 1214B.

FIG. 16 is an enlarged cross-sectional view of vessel assembly 800 of FIG. 11 during a further stage of fenestration of fenestration segment stent-graft 100. Referring now to FIGS. 11 and 16 together, cutting strut catheter 1000 is removed. Accordingly, collateral opening 1112 is open and in alignment with branch vessel 806. In the above manner, collateral opening 1112 if formed in situ to match the particular position of branch vessel 806 thus avoiding custom fabrication of a main stent-graft.

FIG. 17 is an enlarged cross-sectional view of vessel assembly 800 of FIG. 16 during a further stage of fenestration of fenestration segment stent-graft 100. Referring now to FIGS. 16 and 17 together, a branch prosthesis 1714, e.g., a coated stent, is deployed into branch vessel 806 using any one of a number of techniques well known to those of skill in the art. Further, guide wire 902 removed.

Branch prosthesis 1714 is located with collateral opening 1112 and engages fenestration segment stent-graft 100. Fenestration section 110 is stretchable and mobile thus creating a seal around branch prosthesis 1714.

In the example illustrated, branch prosthesis 1714 includes a proximal flange 1716 which engages fenestration section 110 of fenestration segment stent-graft 100. Proximal flange 1716 seals branch prosthesis 1714 to fenestration segment stent-graft 100.

Branch prosthesis 1714 defines a branch lumen 1718 therein. Blood flow flows through branch lumen 1718 of branch prosthesis 1714 thus perfusing branch vessel 806. By providing a sufficient diameter to proximal flange 1716, fenestration section 110 is sufficiently sealed by proximal flange 1716 ensuring blood flows through branch lumen 1718 of branch prosthesis 1714 in contrast through the un-fenestrated portion of fenestration section 110.

In one example, a balloon sheath (a balloon attached to or part of the outside of a sheath (catheter) which when inflated fills the vessel and causes the catheter shaft associated with it to be biased to one side or held in the middle according to the balloon sheath's configuration) is use to aid in the placement and manipulation of branch prosthesis 1714. This balloon sheath is also used to provide active control of perfusion to branch vessel 806. More particularly, inflation of a balloon of the balloon sheath distal to branch vessel 806 increases the pressure differential between main vessel 802 and branch vessel 806 directing more blood flow into branch vessel 806. Further, a perfusion port of a distal side of the balloon can be used to infuse additional anticoagulant medications increasing the relative concentration of the medications at ostia 810, 812 as compared to a systemic application of the medications.

The procedure illustrated and discussed above in reference to FIGS. 9-17 is repeated to form a collateral opening in fenestration section 110 and to deploy a branch prosthesis in alignment with branch vessel 808 and so is not repeated here.

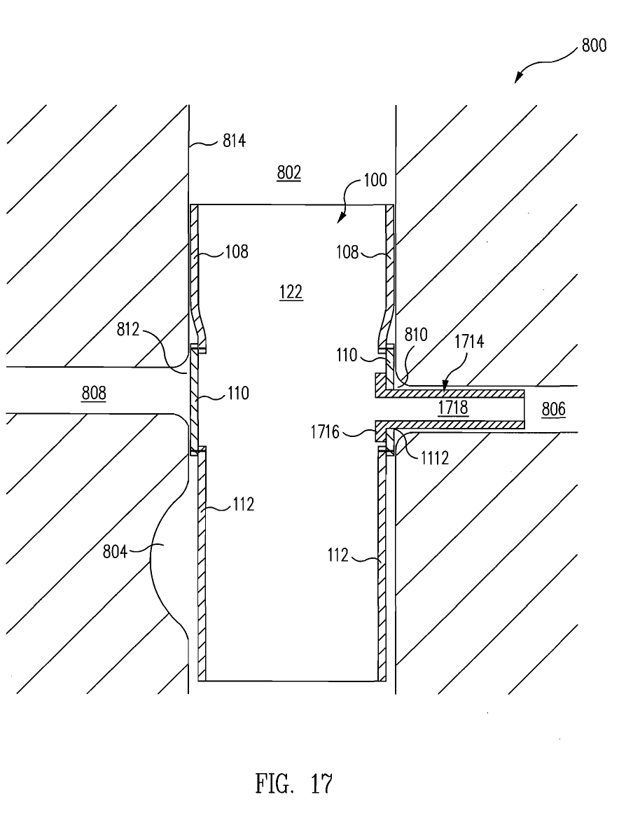

FIG. 18 is a schematic view of a vessel assembly 1800 including a fenestration segment stent-graft 100A similar to fenestration segment stent-graft 100 of FIGS. 1 and 2 prior to fenestration in accordance with another example. Referring now to FIG. 18, a typical abdominal aortic aneurysm (AAA) 1804 is illustrated with the proximal aorta 1802 leading to renal arteries 1806, 1808 and distal iliac arteries 1830, 1832. In accordance with this example, fenestration segment stent-graft 100A is deployed such that a fenestration section 110A is aligned with renal arteries 1806, 1808. A bifurcated endovascular device 1840 is engaged with a distal section 11 2A of fenestration segment stent-graft 100A. Bifurcated endovascular device 1840 includes a main body 1842 and two connected extension portions 1844, 1846 extending into iliac arteries 1830, 1832. In this manner, aneurysm 1804 is excluded.

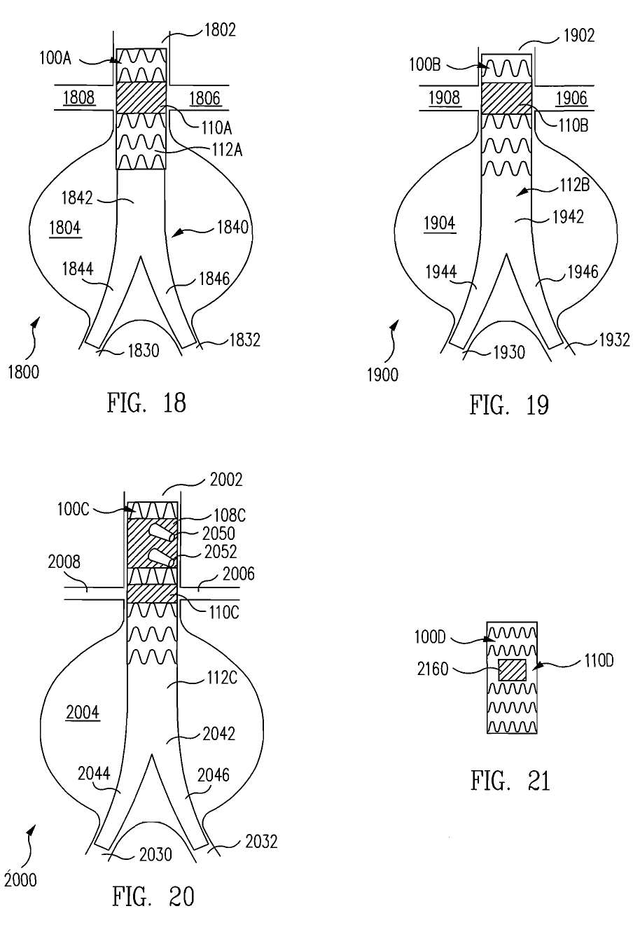

FIG. 19 is a schematic view of a vessel assembly 1900 including a fenestration segment stent-graft 100B similar to fenestration segment stent-graft 100 of FIGS. 1 and 2 prior to fenestration in accordance with another example. Referring now to FIG. 19, a typical abdominal aortic aneurysm (AAA) 1904 is illustrated with the proximal aorta 1902 leading to renal arteries 1906, 1908 and distal iliac arteries 1930, 1932. In accordance with this example, fenestration segment stent-graft 100B is deployed such that a fenestration section 110B is aligned with renal arteries 1906, 1908. A distal section 112B of fenestration segment stent-graft 100B includes a main body 1942 and two extension portions 1944, 1946 extending into iliac arteries 1930, 1932. In this manner, aneurysm 1904 is excluded.

FIG. 20 is a schematic view of a vessel assembly 2000 including a fenestration segment stent-graft 100C similar to fenestration segment stent-graft 100 of FIGS. 1 and 2 prior to fenestration in accordance with another example. Referring now to FIG. 20, a typical abdominal aortic aneurysm (AAA) 2004 is illustrated with the proximal aorta 2002 leading to renal arteries 2006, 2008 and distal iliac arteries 2030, 2032. In accordance with this example, fenestration segment stent-graft 100C is deployed such that a fenestration section 110C is aligned with renal arteries 2006, 2008. A distal section 11 2C of fenestration segment stent-graft 100C includes a main body 2042 and two extension portions 2044, 2046 extending into iliac arteries 2030, 2032. In this manner, aneurysm 2004 is excluded.

Further, a proximal section 108C includes branch prosthesis 2050, 2052 to perfused branch vessels emanating from aorta 2002 such as, for example, the superior mesenteric artery (SMA). In another example, instead of providing branch prosthesis 2050, 2052, a fenestration segment stent-graft similar to fenestration segment stent-graft 100C includes a scallop at the proximal edge of the fenestration segment stent-graft to avoid blocking of the superior mesenteric artery and also to provide a means for aligning the fenestration section with the renal arteries.

FIG. 21 is a schematic view of a fenestration segment stent-graft 100D similar to fenestration segment stent-graft 100 of FIGS. 1 and 2 in accordance with another example. In accordance with this example, a fenestration section 110D includes one or more fenestration regions 2160 (in contrast to a continuous cylinder fenestration section) formed of materials such as those set forth above for fenestration section 110 of fenestration segment stent-graft 100 of FIGS. 1 and 2. The remainder of fenestration section 110D is formed of standard graft material, e.g., woven graft cloth. Stated another way, fenestration section 110D includes woven graft cloth and one or more fenestration regions 2160 formed therein.

Fenestration regions 2160 are windows just large enough to accommodate a desired range of anatomical variations in branch vessel placement. For example, fenestration regions 2160 are 15×15 mm squares that allow for 5 mm off “idealized” renal location in both the circumferential and longitudinal directions. In another example, fenestration regions 2160 are porous strips that accommodate a full range of longitudinal variations of the location of the branch vessels.

This disclosure provides exemplary embodiments. The scope is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. A fenestration segment stent-graft comprising:
a proximal section comprising a woven graft cloth;
a distal section comprising a woven graft cloth; and
a fenestration section attached to and between said proximal section and said distal section, said fenestration section comprising knit cloth, said fenestration section having a greater resistance to tearing than said proximal section and said distal section.

2. The fenestration segment stent-graft of claim 1 wherein said proximal section comprises a proximal end and a distal end, said distal end being attached to a proximal end of said fenestration section by a first attachment means.

3. The fenestration segment stent-graft of claim 2 wherein said attachment means comprises stitching.

4. The fenestration segment stent-graft of claim 2 wherein a distal end of said fenestration section is attached to a proximal end of said distal section by a second attachment means.

5. The fenestration segment stent-graft of claim 4 wherein said second attachment means comprises stitching.

6. The fenestration segment stent-graft of claim 1 wherein said fenestration segment stent-graft defines a main lumen extending generally parallel to a longitudinal axis of said fenestration segment stent-graft and between a proximal main opening and a distal main opening of said fenestration segment stent-graft.

7. The fenestration segment stent-graft of claim 1 wherein said proximal section, said fenestration section, and said distal section have a uniform diameter.

8. The fenestration segment stent-graft of claim 1 further comprising at least one stent ring.

9. The fenestration segment stent-graft of claim 8 wherein said at least one stent ring comprises:
a stent ring attached to a proximal end of said proximal section.

10. The fenestration segment stent-graft of claim 8 wherein said at least one stent ring comprises:
a first stent ring attached to said proximal section; and
at least a second stent ring attached to said distal section.

11. The fenestration segment stent-graft of claim 1 wherein said fenestration section has an absence of stent rings.

12. The fenestration segment stent-graft of claim 1 further comprising a collateral opening in said fenestration section.

13. The fenestration segment stent-graft of claim 1 further comprising a branch prosthesis located within said collateral opening.

14. The fenestration segment stent-graft of claim 13 wherein said branch prosthesis comprises a proximal flange that engages said fenestration section.

15. The fenestration segment stent-graft of claim 1 further comprising a bifurcated endovascular device coupled to said distal section, said bifurcated endovascular device comprising:
a main body; and
two extension portions connected to said main body.

16. The fenestration segment stent-graft of claim 1 wherein said distal section comprises:
a main body; and
two extension portions connected to said main body.

17. The fenestration segment stent-graft of claim 16 wherein said proximal section comprises at least one branch prosthesis.

18. The fenestration segment stent-graft of claim 1 wherein said fenestration section comprises:
woven graft material; and
one or more fenestration regions comprising said knit cloth formed in said woven graft material of said fenestration section.

19. The fenestration segment stent-graft of claim 1 wherein said knit cloth is impregnated with an elastomer.

20. A fenestration segment stent-graft comprising:
a proximal section;
a distal section; and
a fenestration section attached to and between said proximal section and said distal section, said fenestration section having a greater resistance to tearing than said proximal section and said distal section.

21. The fenestration segment stent-graft of claim 20 wherein said fenestration section comprises:
graft material comprising loose woven fibers.

22. The fenestration segment stent-graft of claim 21 wherein said loose woven fibers are continuous.

23. The fenestration segment stent-graft of claim 21 wherein said graft material further comprises velour.

24. The fenestration segment stent-graft of claim 20 wherein said fenestration section comprises:
graft material comprising loose random fibers.

25. The fenestration segment stent-graft of claim 24 wherein said graft material further comprises velour.

26. The fenestration segment stent-graft of claim 20 wherein said fenestration section comprises:
graft material comprising a tubular braid.

27. The fenestration segment stent-graft of claim 20 wherein said fenestration section comprises:
graft material comprising low density monofilament graft material.

28. The fenestration segment stent-graft of claim 27 further comprising an anti-thrombogenic coating applied to said low density monofilament graft material.

29. The fenestration segment stent-graft of claim 28 further comprising a thrombogenic coating applied to said low density monofilament graft material, said anti-thrombogenic coating applied to said thrombogenic coating.

30. The fenestration segment stent-graft of claim 27 further comprising a thrombogenic coating applied to said low density monofilament graft material.

31. A method comprising:
deploying a fenestration segment stent-graft into a main vessel such that a fenestration section of said fenestration segment stent-graft covers a first branch vessel emanating from said main vessel, said fenestration segment stent-graft comprising:
a proximal section;
a distal section; and
said fenestration section attached to and between said proximal section and said distal section, said fenestration section having a greater resistance to tearing than said proximal section and said distal section.

32. The method of claim 31 further comprising:
forming a guidewire hole in said fenestration section aligned with said branch vessel.

33. The method of claim 32 further comprising:
dilating said guidewire hole to form a dilated guidewire hole.

34. The method of claim 33 wherein said dilating comprises inserting a cutting strut catheter into said guidewire hole.

35. The method of claim 34 further comprising retracting an outer sheath of said cutting strut catheter to expose a cutting strut device of said cutting strut catheter; and
expanding said cutting strut device to enlarge said dilated guidewire hole into a collateral opening.

36. The method of claim 35 wherein said cutting strut device comprises struts comprising sharp edges.

37. The method of claim 35 further comprising deploying a branch prosthesis in said collateral opening.
